(19) United States
(12) Patent Application Publication
(10) Pub. No.: US 2023/0146265 A1
(43) Pub. Date: May 11, 2023

(54) LYSIS OF A SAMPLE BY MEANS OF MAGNETIC ELEMENTS AND ROTATIONAL RELATIVE MOVEMENT

(71) Applicants: Hahn-Schickard-Gesellschaft für angewandte Forschung e.V., Villingen-Schwenningen (DE); Spindiag GmbH, Freiburg (DE)

(72) Inventors: Judith SCHLANDERER, Freiburg (DE); Nils PAUST, Freiburg im Breisgau (DE); Frank SCHWEMMER, Freiburg (DE); Gregor CZILWIK, Freiburg (DE)

(21) Appl. No.: 18/153,768

(22) Filed: Jan. 12, 2023

Related U.S. Application Data
(63) Continuation of application No. PCT/EP2021/069461, filed on Jul. 13, 2021.

(30) Foreign Application Priority Data
Jul. 17, 2020 (DE) 10 2020 209 001.7

(51) Int. Cl. C12M 1/00; C12N 1/06

(52) U.S. Cl. CPC C12M 47/06; C12N 1/066

(57) ABSTRACT

Lysis apparatus including a chamber for receiving a sample and at least one magnetic actuator located within the chamber as well as at least two magnetic elements arranged outside the chamber. Above that, such a lysis apparatus includes a driving unit for effecting a rotational relative movement between the chamber and the at least two magnetic elements arranged outside the chamber, wherein the polarity of the magnetic elements is opposite with respect to the circular path of the rotational relative movement and hence, for example, the chamber, such that the magnetic actuator arranged within the chamber is moved both translationally and rotationally to effect lysis of the sample. Here, the chamber is configured, e.g., by its dimensioning or a flexible outer shell, to enable the at least one magnetic actuator located within the chamber to move both translationally and rotationally.

### CROSS-REFERENCE TO RELATED APPLICATIONS

This application is a continuation of copending International Application No. PCT/EP2021/069461, filed Jul. 13, 2021, which is incorporated herein by reference in its entirety, and additionally claims priority from German Application No. 10 2020 209 001.7, filed Jul. 17, 2020, which is also incorporated herein by reference in its entirety.

The present disclosure relates to apparatuses and methods for lysis of a sample by means of magnetic elements and rotational relative movement and in particular to respective apparatuses and methods in the field of centrifugal microfluidics.

### BACKGROUND OF THE INVENTION

In biology and medicine, microorganisms are opened up for research purposes by mechanical friction, impact and shear forces in order to reach the interior of the microorganisms. For example, cells can be actively broken up to reach proteins and/or DNA inside the cell. Such opening up of microorganisms is also referred to as lysis. Apparatuses and methods for effecting such opening up of microorganisms can thus be referred to as lysis apparatuses and lysis methods.

Different methods for opening up microorganisms by mechanical friction, impact and shear forces are known.

Methods with translational, radial movement of magnetic actuators in centrifugal microfluidic systems can be found in Kido et al. [1] and CA 2827614 C. Kido et al. [1] describe a centrifugal microfluidic method for cell lysis, where shear and friction forces are realized by translational, radial movement of the magnetic actuator. In CA 2827614 C, a disc-shaped actuator is also moved in translational, radial movement by external magnetic forces within a chamber. The functionality of both approaches is similar.

A method with translational azimuthal movement of magnetic actuators in a centrifugal microfluidic system is described by Siegrist et. al [2]. In contrary to the systems described so far, Siegrist et. al [2] disclose a centrifugal microfluidic method where the friction of the glass particles is realized by a translational azimuthal movement.

A method with translational movement of magnetic actuators in a non-centrifugal microfluidic system is disclosed in the U.S. Pat. No. 8,356,763 B2. U.S. Pat. No. 8,356,763 B2 describes, as a non-centrifugal microfluidic lysis system, a system generating the magnetic actuation by switching on, off and over electromagnets. Here, the magnetic actuator is moved translationally, a rotational movement is prevented by the chamber.

A method with translational and rotational movement of magnetic actuator in an non-centrifugal microfluidic system is disclosed in U.S. Ser. No. 10/138,458 B2, where a method for lysis of cells is described. Rotating external magnets generate a varying magnetic field that imparts rotational movements, translational movements or a combination of these movements to the magnetic actuator.

The inventors have found out that the methods for mechanical lysis of microorganisms known from conventional technology suffer from several disadvantages. For example, none of the centrifugal microfluidic approaches uses all possible degrees of freedom for moving the magnetic actuator. Therefore, the potential of possible collisions between magnetic actuator, particles and microorganisms is not fully exploited. In the non-centrifugal system as described in U.S. Ser. No. 10/138,458 B2, the degrees of freedom are partly exploited. However, this involves a complicated structure that can be used exclusively for the step of lysis of microorganisms. The handling of all further steps has to take place manually.

### SUMMARY

According to an embodiment, a lysis apparatus may have: a chamber for receiving a sample; at least one magnetic actuator located within the chamber; at least two magnetic elements arranged outside the chamber and driving means for effecting a rotational relative movement between the chamber and the magnetic elements arranged outside the chamber, by which the chamber successively passes the magnetic elements located outside the chamber, wherein the polarity of the magnetic elements is opposite with respect to the circular path of the rotational relative movement, such that the magnetic actuator arranged within the chamber is moved both translationally and rotationally around an own axis of the magnetic actuator to effect lysis of the sample, wherein the chamber is configured to enable the at least one magnetic actuator located within the chamber to move both translationally and rotationally around an own axis of the magnetic actuator.

According to another embodiment, a lysis method may have the steps of: introducing a sample into a chamber, wherein at least one magnetic actuator is located in the chamber and wherein the chamber is configured to enable the magnetic actuator located within the chamber to move both translationally and rotationally around an own axis of the magnetic actuator; effecting a rotational relative movement between the chamber wherein the sample and at least one magnetic actuator are located, and between at least two magnetic elements located outside the chamber, wherein the polarity of the at least two magnetic elements outside the chamber is opposite with respect to the circular path of the rotational relative movement, such that the magnetic actuator located within the chamber is moved both translationally and rotationally around an own axis of the magnetic actuator to effect lysis of the sample.

Examples of the present disclosure provide a lysis apparatus comprising a chamber for receiving a sample and at least one magnetic actuator located within the chamber, as well as at least two magnetic elements located outside the chamber that can be configured, for example, as permanent magnets or electromagnets. Here, for example, the chamber can be a lysis chamber and/or part of a fluidic module or a cartridge. The magnetic actuator within the chamber for example, can be a lysis chamber and/or part of a fluidic module or a cartridge. The magnetic actuator within the chamber can be configured, for example, as permanent magnet. Above that, such a lysis apparatus comprises driving means for effecting a rotational relative movement between the chamber and the at least two magnetic elements arranged outside the chamber, wherein the polarity of the magnetic elements is opposite with respect to the circular path of the rotational relative movement and hence the chamber, such that the magnetic actuator arranged within the chamber is moved both translationally and rotationally to effect lysis of the sample. Here, the chamber is configured, for example, by its dimensioning or a flexible outer shell, to enable the at least one magnetic actuator located within the chamber to move both translationally and rotationally.

Examples of the present disclosure provide a lysis method, wherein a sample is introduced into a chamber or a lysis chamber wherein at least one magnetic actuator is located within the chamber, which is configured, for example, as permanent magnet, and wherein the chamber is configured, for example, by its dimensioning or a flexible outer shell, to enable the at least one magnetic actuator located within the chamber to move both translationally and rotationally. Here, the chamber can be, for example, part of a fluidic module or a cartridge. In the method, driving means effect a rotational relative movement between the chamber, where the sample and at least one magnetic actuator are located, and at least two magnetic elements arranged outside the chamber, wherein the polarity of the at least two magnetic elements outside the chamber is opposite with respect to the circular path of the rotational relative movement and hence the chamber, such that the magnetic actuator arranged in the chamber is moved both translationally and rotationally to effect lysis of the sample. The at least two magnetic elements arranged outside the chamber can be configured as permanent magnets or electromagnets.

Examples of the present disclosure are based on the core idea of moving the magnetic actuator within the chamber both translationally and rotationally in a centrifugal mechanical lysis apparatus, with the help of a rotational relative movement between at least two magnetic elements outside a chamber and the chamber that includes a sample to be lysed and at least one magnetic actuator. It has been found that the translation and rotation of the magnetic actuator within the chamber is enabled by the polarity of the at least two magnetic elements outside the chamber, in that the at least two magnetic elements outside the chamber are oppositely polarized with respect to the circular path of the rotational relative movement and hence the chamber. This can have the effect that the magnetic actuator within the chamber does not only perform translation but also rotation. In examples, the rotational relative movement is effected in that the chamber rotates around an axis of rotation. In examples, the rotational relative movement is effected in that the two magnets outside the chamber rotate around the same axis of rotation.

In examples, by using all degrees of freedom of movement of the magnetic actuator within the chamber, for example samples that are difficult to lyse can be lysed with little time effort. By the more efficient lysis, for example, reduction of the installation space of such a lysis apparatus can be realized. By using all degrees of freedoms in a centrifugal mechanical apparatus, this efficient form of the lysis can be integrated, for example, in a simple manner into an apparatus that is configured to perform further sample preparation steps and/or sample analysis steps.

In examples, the magnetic elements are configured as magnetic poles. In examples, the magnetic elements are individual magnetic poles in that the lysis apparatus comprises magnets outside the chamber, wherein only one magnetic pole each has a significant influence on the magnetic actuator within the rotating chamber, such that the influence of further poles of the magnets outside the chamber, normally the influence of the second pole of each magnet outside the chamber, can be neglected for the magnetic actuator within the chamber. By such an arrangement, the structure of specific magnetic field profiles that can be favorable for specific lysis applications can be allowed. In examples, reducing the installation space requirement in radial direction with respect to the rotational plane of the chamber can be obtained, for example when rod magnets are arranged perpendicular to the plane of rotation, such that the respective second pole of the same is sufficiently far apart from the rotating chamber such that its respective influence can be neglected. In examples, the magnetic elements can be the poles of bent magnets, wherein each pole of the bent magnets each forms one magnetic element. The rest of the bent magnet, for example horseshoe magnet, can be located, for example, above or below the plane of rotation of the chamber.

In examples, the magnetic elements are configured as magnets. In such examples, a particularly simple structure can be possible as there are no restrictions, for example, with respect to the influence of a second magnetic pole or, for example, no additional installation space requirement by bent magnets as in the configurations of the magnetic elements as magnetic poles. Above that, examples where each magnetic element is a magnet allow the structure of specific magnetic field profiles, which can be favorable for specific lysis applications.

In examples, the lysis apparatus is configured to at least reduce the magnetic field acting on the magnetic actuator arranged within the chamber from the at least two magnetic elements arranged outside the chamber, independent of the rotational relative movement. Thereby, it is, for example, possible to stop the lysis without stopping the rotational relative movement. This type of switching-off can be advantageous, for example, when the rotational relative movement is needed for further process steps.

In examples, the lysis apparatus comprises actuating means to a change the distance between the chamber and the at least two magnetic elements arranged outside the chamber independent of the rotational relative movement. This can take place, for example, by a translational relative movement between the chamber and the magnets arranged outside the same. Thereby, it is possible, for example, to stop the lysis by a simple translation of the at least two magnetic elements arranged outside the chamber without stopping the rotational relative movement. This type of switching-off is, for example, easy to implement and can be advantageous, for example, when the rotational relative movement is needed for further process steps and should therefore not be stopped.

In examples, the lysis apparatus comprises actuating means that are configured to move the at least two magnetic elements arranged outside the chamber perpendicular to the plane of rotation, such that, for example, their influence on the lysis can be reduced or increased, such that, for example, the lysis can be stopped or started. Thus, for example, only a small installation space is needed in radial direction for implementing the actuator system for moving the at least two magnetic elements. Thereby, for example, overall integration into an apparatus configured to perform further sample preparation and analysis steps can be simplified.

In examples, the lysis apparatus comprises actuating means configured to move the at least two magnetic elements arranged outside the chamber in parallel to the plane of rotation, independent of the rotational relative movement, such that, for example, the lysis can be stopped without stopping the rotational relative movement. In such examples, for moving the at least two magnetic elements away from an axis of rotation, the centrifugal force can be used, for example when the magnetic elements arranged outside the chamber are spring-mounted and rotate around the same axis of rotation. For example, by spring-mounting, a force can be applied on both magnetic elements that acts towards the axis of rotation, such that by increasing the rotational speed, the magnetic elements can be moved away from the axis of rotation against the spring force. This movement away from the axis of rotation and towards the axis of rotation is also a translational movement, independent of the rotational relative movement.

In examples, the at least two magnetic elements arranged outside the chamber can be controllable and/or variable electromagnets such that, for example, their influence on the lysis can be reduced or increased. For example, starting or stopping the lysis can be caused by the electromagnets independent of the rotational relative movements, such that further process steps can be performed before or after without needing a further actuator system for moving the at least two magnets outside the chamber. Additionally, the lysis apparatus can be configured with a lower installation space requirement.

In examples, the lysis apparatus comprises at least one and usually several lysis particles located within the chamber, for example microparticles, spherical microparticles or beads. The at least one lysis particle can consist, for example, of glass, silica zirconia, zirconia, metal or other ceramics and glass materials. In examples, the at least one lysis particle can have, for example, maximum dimensions of less than 0.5 mm. Here, the at least one lysis particle eases the lysis of the samples as the mechanical effects on the sample in the chamber are increased by additional particles.

In examples, the at least two magnetic elements arranged outside the chamber are stationary at the time of the lysis and the driving means are configured to rotate the chamber with respect to an axis of rotation relative to the magnetic elements arranged outside the chamber. Thereby, for example, a very simple structure of the lysis apparatus and a simple integration into an apparatus that is configured to perform further sample preparation and analysis steps can be obtained, since only the chamber rotates and no rotational actuator system is needed for the at least two magnetic elements outside the chamber.

In examples, a diaphragm, for example a filter diaphragm or a sterile filter, is located within the chamber, which allows enriching microorganisms from a greater volume and subsequent lysis on the diaphragm.

In examples, the lysis apparatus comprises a tempering means that is configured to change the temperature of the chamber, for example to heat the same, for example, to a temperature of 120° C.

Here, the tempering means can be configured as contact heating. Thereby the mechanical lysis can be supported by a thermal input.

In examples, the at least two magnetic elements outside the chamber are arranged at an angle in the plane of rotation between 20° and 180° to one another. The angle is formed between lines connecting the respective centers of the magnetic elements to the axis of rotation. Thereby, suitable profiles of the magnetic field can be generated, by which the magnetic actuator can be moved rotationally and translationally within the chamber.

In examples, the rotational relative movement is performed in a rotational frequency range of 0.5 Hz to 40 Hz, advantageously 2 Hz to 30 Hz. By choosing the frequency range, for example, an efficient lysis having a sufficiently good and at the same time fast result can be performed.

In examples, the chamber comprises, in two of three spatial directions that are perpendicular to one another, at least the length of the longest diagonal of the magnetic actuator located within the chamber, and in a third of the three spatial directions that are perpendicular to the one another, at least the length of the longest diagonal of the magnetic actuator located within the chamber minus 20%. In examples, the chamber comprises, in three of three spatial directions that are perpendicular to one another, at least the length of the longest diagonal of the magnetic actuator located within the chamber. By such a dimensioning of the chamber, for example, free movement of the magnetic actuator within the chamber can be enabled, or, for example, a defined limited movement of the magnetic actuator, which can be particularly favorable for the lysis.

In examples, the chamber comprises, in at least two directions of three directions that are formed by the direction of the axis of rotation, the radial direction with respect to the rotation and the azimuthal direction with respect to the rotation, at least the size of the length of the magnetic actuator located within the chamber. By such a dimensioning of the chamber, for example, a specific type of rotation can be enabled, for example to enable efficient lysis. This dimensioning can also allow, for example, an advantageous design of the apparatus.

In examples, the at least two magnetic elements outside the chamber comprise, at the time of the lysis, a maximum perpendicular distance of 5 cm to the chamber with respect to the plane of the rotational relative movement. In examples, the at least two magnetic elements outside the chamber comprise, at the time of the lysis, a maximum radial distance from the actuator located within the chamber of 5 cm. By such distances of the magnetic elements and the magnetic actuator or the magnetic elements and the chamber, for example, efficient lysis can be enabled and at the same time, for example, only a small insulation space is needed for such an apparatus.

In examples, the lysis apparatus comprises tempering means configured to heat the at least one magnetic actuator located within the chamber above its Curie temperature in order to inactivate the magnet. Thereby, for example, an option of stopping the lysis can be provided, independent of the rotational relative movement or an actuator system for the at least two magnets located outside the chamber or their possible configuration as electromagnets. By this option, for example a particularly small design of the lysis apparatus can be obtained. In addition, for example, inactivation of the magnetic actuator within the chamber can be favorable for further process steps.

In examples, the at least two magnetic elements arranged outside the chamber are moved independent of the rotational relative movement in order to at least reduce the magnetic field acting on the magnetic actuator arranged within the chamber. Thereby, it is, for example, possible to stop the lysis without interrupting the rotational relative movement, which is needed, for example, for further process steps, such that, for example, a simpler integration into a process chain of sample preparation and analysis is possible.

In examples, the at least two controllable and/or variable electromagnets arranged outside the chamber are controlled or regulated such that the magnetic field acting on the magnetic actuator arranged within the chamber is at least reduced, independent of the rotational relative movement. Thereby, it is, for example, possible to stop the lysis without interrupting the rotational relative movement, which is needed, for example, for further process steps, such that, for example, a simpler integration into a process chain of sample preparation and analysis is possible. In addition, for example, no further actuator system is mandatory for moving the at least two magnetic elements outside the chamber. Thereby, for example, the lysis apparatus can be configured with less installation space requirements with the same functionality compared to the implementation by means of permanent magnets.

Examples of the present disclosure relate to a method for efficient lysis of microorganisms of complex samples.

In examples, spherical microparticles, a magnetic actuator and the samples are located within a lysis chamber of a centrifugal microfluidic cartridge. At least two permanent magnets or electromagnets are statically arranged, for example, above or below the cartridge. By rotating the cartridge, a constantly changing magnetic field can be generated in the lysis chamber. Thereby, the actuator can be set in motion strongly and the same can move both translationally to the closest magnet and rotationally around at least one of its own axes. In combination with microparticles, strong friction, impact and shear movements can result in the lysis chamber.

In order to obtain both translation and rotation of the magnetic actuator simultaneously, the lysis chamber can be configured to comprise at least the size of the length of the magnetic actuator in two spatial directions. Further, the at least two external magnetic elements, i.e., located outside the lysis chamber, have an angle with respect to each other and are arranged with opposite polarity in relation to the lysis chamber.

Examples of the present disclosure are hence configured to efficiently open up, i.e., to lyse, bacteria, yeasts, viruses, fungi, spores or other microorganisms within the sample in a shortest possible time.

Examples of the present disclosure relate to a method for mechanical lysis on a centrifugal microfluidic structure. Examples allow, with minimum handling effort, to provide an efficient lysis where even microorganisms that are difficult to lyse can be handled. As one of the core steps in sample preparation, an efficient lysis allows a highly sensitive molecular diagnostic identification, for example by means of qPCR (quantitative polymerase chain reaction).

In examples, rotational and translational movements of a magnetic actuator are effected due to rotation of a fluidic module or a cartridge by an external static magnetic field.

Further examples include lysis apparatuses, wherein the polarity of the magnetic elements outside the chamber, for example the external magnets, is opposite to one another with respect to lysis chamber to initiate a rotational movement of the magnetic actuator.

### BRIEF DESCRIPTION OF THE DRAWINGS

Embodiments of the present invention will be detailed subsequently referring to the appended drawings, in which:

FIG. 1a is a schematic top view of an example of a lysis apparatus;

FIG. 1b is a schematic side view of the lysis apparatus of FIG. 1a;

FIG. 2 is a schematic top view of a further example of a lysis apparatus where the magnets are arranged at a different angle;

FIG. 3 a schematic top view of a further example of a lysis apparatus with a different arrangement of the magnet;

FIG. 4 is a schematic top view of an example of a lysis apparatus with a further different arrangement of magnetic elements;

FIG. 5 are schematic side views of two examples of a lysis apparatus with actuating means for influencing the magnetic field on the magnetic actuator independent of the rotational relative movement;

FIG. 6 is a plot as a comparison of a qPCR analysis of a lysate according to the present disclosure and a thermal reference lysate;

FIG. 7 is a schematic side view of an example of a chamber of a lysis apparatus with a diaphragm;

FIG. 8 is a schematic side view of an example of a lysis apparatus with a tempering means.

### DETAILED DESCRIPTION OF THE INVENTION

In the following, examples of the present disclosure are described in detail and using the attached drawings. It should be noted that the same elements or elements having the same functionality are provided with the same or similar reference numbers, wherein a repeated description of elements that are provided with the same or similar reference numbers is typically omitted. In particular, same or similar elements can each be provided with reference numbers having a same number with a different or no small letter. Descriptions of elements that have the same or similar reference numbers are inter-exchangeable. In the following description, many details are described to provide a substantial explanation of examples of the disclosure. However, it is obvious for a person skilled in the art that other examples can be implemented without these specific details. Features of the different described examples can be combined except when features of a corresponding combination exclude each other or such a combination is explicitly excluded.

Before examples of the present disclosure are explained in more detail, definitions of some of the terms used herein are indicated.

Cartridge: Cartridges are disposable parts of polymer including channels, chambers for guiding, processing and analyzing samples. Further, cartridges can include interfaces for the introduction of samples and potentially the extraction of liquids.

Sample: Introduce substance (frequently a liquid) including microorganisms.

Lysis: Destruction of a cell by damaging the outer cell membrane.

Microparticles/beads as examples of lysis particles: Spherical elements with a typical diameter of 0.1 mm-3 mm of, for example, glass, silica zirconia, zirconia, metal or other ceramics and glass materials.

Magnetic actuator: Small co-rotating magnet, e.g., rod magnet set into motion by an external magnetic field.

Lysis chamber: A chamber, for example on a cartridge where the lysis process is performed includes, for example, particles and at least one magnetic actuator.

External magnet: Permanent magnet or electromagnet that can, for example, be statically arranged, for example, outside the chamber, e.g., above or below the cartridge.

Polarity of two magnets: Orientation of north and south pole with respect to (Δr, Δφ, for example radial and azimuthal with respect to a rotation center)—expansion of the lysis chamber, for example with respect to the circular path of a rotational relative movement between the chamber and the magnetic elements outside the chamber.

Rotational movement: Describes the movement, for example, of a magnetic actuator around at least one of its own axes, e.g., the own axis.

Translational Movement: Describes the same displacement of all points of a rigid body, for example a magnetic actuator, at a given time. Velocity and acceleration of all points are identical and they move on parallel trajectories.

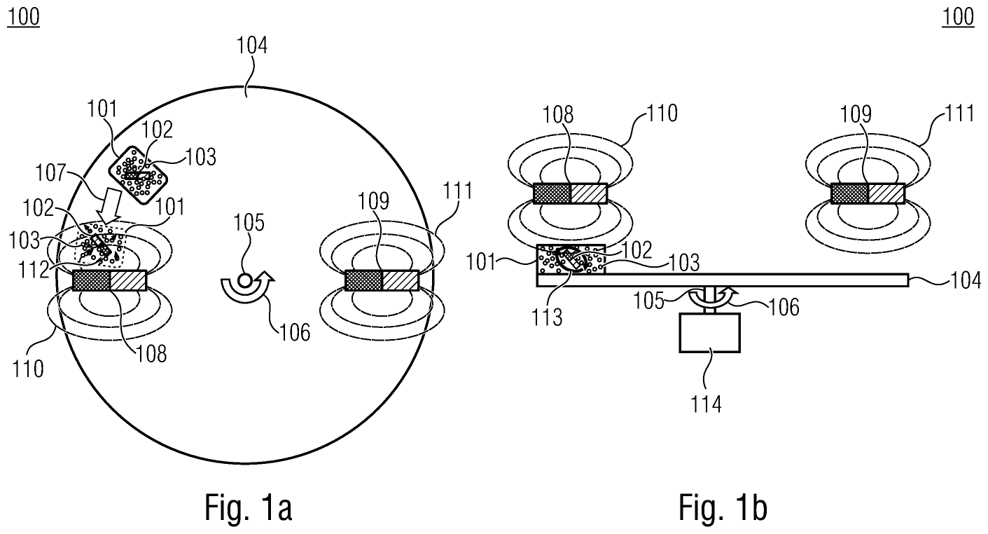

FIG. 1a shows a schematic top view and FIG. 1b a schematic side view of an example of a lysis apparatus. The illustrated lysis apparatus 100 consists of a chamber 101 including a magnetic actuator 102 and lysis particles 103. In the shown example, the magnetic actuator 102 is a rod magnet. The chamber is mounted on a carrier 104. The carrier 104 and hence the chamber 101 are configured to rotate around an axis of rotation 105. The rotational movement of the carrier is indicated by an arrow 106. The resulting rotational movement of the chamber on a circular path is indicated by a further arrow 107. In this example, two static magnetic elements 108, 109 configured as magnets are located above the chamber, i.e., perpendicular to the plane of rotation. The magnetic field of the magnetic element 101 is indicated by a field line profile 110, the magnetic field of the magnetic element 109 by the field line profile 111. The magnetic elements 108, 109 are oriented orthogonally to the rotational circular path of the chamber with respect to the connecting line of their respective north and south poles (magnetic axis). Above that, the two magnetic elements 108, 109 have an opposite polarity with respect to the rotational circular path of the chamber. The opposite polarity will be discussed in more detail in connection with the following description of the functionality. The magnetic elements are arranged at an angle of 180° to each other with respect to the circular path. The chamber is indicated in dotted lines at a second time. This shows the movement of the chamber 101 on the rotational circular path around the axis of rotation 105. Within the moved chamber 101, the moving magnetic actuator 102 is illustrated also at a second time in amended position and orientation compared to the magnetic actuator prior to the movement. The moved lysis particles 103 at the second time are also illustrated in the moved chamber 101. The movement of the lysis particles themselves due to the rotation of the chamber 101, as well as due to the movement of the magnetic actuator 102, is indicated by arrows 112. Further, the rotational movement of the magnetic actuator during the rotation of the chamber is indicated by arrows 113 in FIG. 1b. For effecting the rotational movement of the carrier 104, a driving means 114 is illustrated at the axis of rotation 105.

In examples, during operation, the chamber 101 rotates on a rotational circular path with respect to the axis of rotation 105. During the rotation, the moved chamber 101 approaches the magnetic element 101. The magnetic field of the magnetic element 110 causes an interaction with the magnetic actuator within the chamber. The magnetic actuator 102 experiences a translational movement due to the magnetic attraction and is oriented by a rotational movement according to the field 110 of the magnetic element 108. By the rotation of the chamber 101 with respect to the axis of rotation 105, however, the magnetic actuator 102 within the chamber has only a very short retention period in the immediate vicinity of the first magnetic element 108. By the rotation, the chamber will approach the next magnetic element 109. Here, with respect to the rotational circular path of the chamber, the polarity of the magnetic element 109 is opposite to the magnetic element 108 passed before by the chamber 101.

Simply put: If one co-rotates mentally with the chamber 101 on the circular path, just before passing the first magnetic element 108 in the direction of rotation with respect to the chamber, the north pole is on the right side and the south pole on the left side of the magnetic element 101. When rotating further, the chamber 101 impinges on the second magnetic element 109. In the direction of its rotation, seen from the chamber, the south pole of the magnetic element 109 is on the right side and the north pole on the left side.

Accordingly, a magnetic actuator 102 located within the chamber 101 experiences, during the rotation of the chamber with respect to the circular path of the chamber, oppositely orientated magnetic fields 101, 111 of respective successive oppositely polarized magnetic elements 108, 109. Due to the successive oppositely polarized magnetic elements 108, 109 with respect to the rotational circular path of the chamber, the above-described rotational movement of the magnetic actuator 102 results in a rotation 113 of the actuator 102 within the chamber. Here, the rotation is also influenced by the translational movement and the inertia of the magnetic actuator. In other words: the magnetic actuator 102 performs a translational movement within the chamber due to the magnetic attraction to the magnetic elements 108, 109 and additionally the magnetic actuator 102 rotates 113 within the chamber due to the rotational relative movement between the chamber 101 and the magnetic elements 108, 109 that are oppositely polarized with respect to the rotational circular path of the chamber.

By the rotation 113 and translation of the magnetic actuator 102, the sample is lysed, in particular with the help of the lysis particles 103 set into motion 112 by the magnetic actuator.

The carrier 104 can be a centrifugal microfluidic test carrier as is known, for example, under the term LabDisk or LabDisk structure or it can be a cartridge or a fluidic module. Such a carrier includes, for example, a lysis chamber in which a magnetic actuator, particles as well as the sample are located. For example, two stationary magnets polarized oppositely relative to the lysis chamber can be positioned above the carrier. By the rotation of a carrier, for example a cartridge, the magnetic actuator can be set in rotation and translation, the particles can thereby be strongly mixed and, for example, bacteria in the sample can be lysed.

In examples, the chamber 101 can also be part of the carrier 104 or can be integrated in the carrier, for example. The magnetic actuator 102 can be configured, for example, as rod magnet. The magnetic elements 108, 109 can be configured as static magnetic elements, such that merely the chamber 101 rotates on a circular path with respect to an axis of rotation 105. However, it is also possible that both the chamber 101 and the magnetic elements 108, 109 rotate with respect to the same axis of rotation 105, or that the chamber is static and only the magnetic elements 108, 109 relate with respect to an axis of rotation 105. Accordingly, the driving means 114 is also to be considered not only as an exclusive drive of the rotation of the chamber 101. A respective usage of the driving means for introducing a rotation of the at least two magnetic elements 108, 109 outside the chamber around the same axis of rotation 105 is also possible, either additionally or exclusively.

Alternatively, the magnetic elements 108, 109 can also be located below the chamber 101 or beside the carrier 104 and hence the chamber 101 or outside the circle of the rotational movement of the chamber 101. The magnetic elements outside the chamber can be configured, for example, as permanent magnets or electromagnets or as single poles of the magnets. Above that, more than two magnetic elements can be arranged outside the chamber. The opposite polarity of the magnetic elements is then to be considered for two successive magnetic elements each with respect to the rotational relative movement of the chamber in the direction of movement of the chamber.

Above that, in examples, more than one magnetic actuator can be located in the chamber.

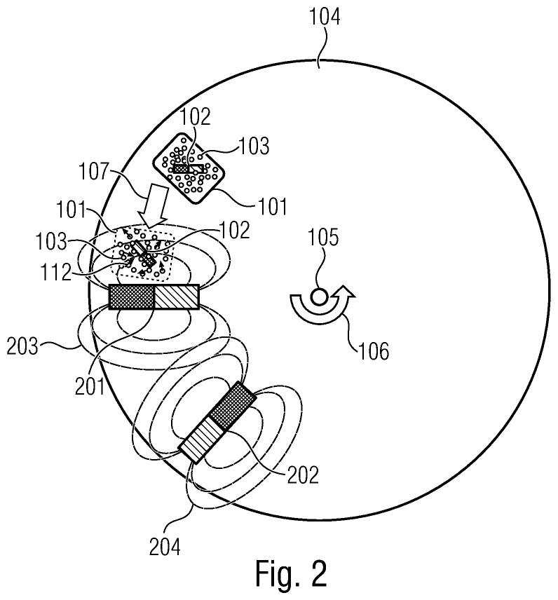

FIG. 2 shows a schematic top view of an example of a lysis apparatus, wherein the magnetic elements are arranged at a different angle.

The illustration corresponds to FIG. 1a, with the exception of the arrangement of the magnetic elements in the form of rod magnets outside the chamber with respect to their angle to one another. Therefore, the same elements are provided with the same reference numbers. The example includes two magnetic elements 201, 202 whose magnetic fields are indicated by magnetic field lines 203 for the magnetic element 201 and magnetic field lines 204 for the magnetic element 202. Compared to FIG. 1a, the two magnetic elements 201, 202 are illustrated in a different angular arrangement to one another. Here, the angle is formed between lines connecting the respective centers of the magnetic elements with the axis of rotation. In the shown example, the angle is approximately 55°. It has shown that an effective lysis can be obtained when the angle is in an angular range of 20° to 180°.

Here, the mode of operation is analog to FIG. 1a. Due to the rotation of the carrier 104, the chamber 101 mounted on the carrier 104 passes the first of the two magnetic elements 201, such that the north pole of the first magnetic element is on the right from the chamber 101 and the south pole on the left, with respect to the circular path of the rotation of the chamber in the direction of movement of the chamber, and when passing the second magnetic element, its polarity is opposite to the first magnetic element, i.e., the north pole of the second magnetic element 202 is correspondingly on the left from the chamber 101 and the south pole on the right, with respect to the circular path of the rotation of the chamber in the direction of movement of the chamber. Thereby, the magnetic actuator experiences rotation and translation, such that effective lysis of the sample is possible.

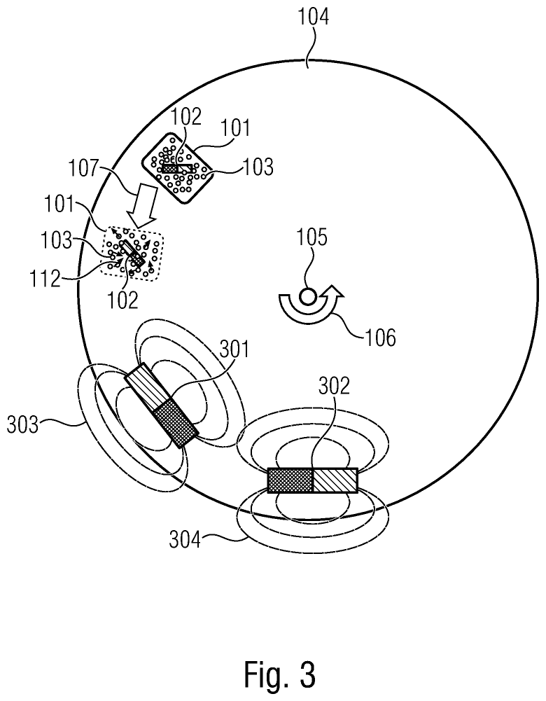

FIG. 3 shows a schematic top view of an example of a lysis apparatus, wherein the magnetic elements in the form of rod magnets have a different orientation with respect to the circular path. The illustration corresponds to FIG. 1a, with the exception of the arrangement of the magnetic elements outside the chamber. Therefore, the same elements are provided with the same reference numbers. The example includes two magnetic elements 301, 302, whose magnetic fields are indicated by magnetic field lines 303 for the magnetic element 301 and magnetic field lines 304 for the magnetic element 302. Compared to FIG. 1a, the magnetic elements 301, 302 have both a different angular arrangement and a different position with respect to the circular path. Compared to FIG. 1a, the magnetic elements 301, 302 are illustrated rotated by 90° in addition to the amended angular arrangement, such that the connecting line between north and south pole of a magnetic element (magnetic axis) is tangential to the rotational circular path of the chamber.

Thereby, FIG. 3 is to illustrate a further possible embodiment of the oppositely polarized magnetic elements. By the rotation of the carrier 104, the chamber 101 mounted on the carrier passes the first magnetic element 301. With respect to its circular path, the chamber 101 encounters first, due to the rotation with respect to the first magnetic element 301, the south pole and subsequently the north pole of the same. When passing the second magnetic element 302 located outside the chamber, the polarity is opposite to the polarity of the first magnetic element 301. Regarding its circular path, the chamber 101 encounters first, due the rotation with respect to the second magnetic element 302, the north pole and subsequently the south pole of the same, i.e., exactly opposite to the first magnetic element 301. The magnetic actuator 102 within the chamber experiences a translational movement due to the magnetic attraction to the magnetic elements 301, 302. When passing the first magnetic element, by the rotation of the chamber, the actuator 102 will orient itself according to the magnetic field of the first magnetic element 303 by a rotation. By the rotation of the chamber, the magnetic actuator subsequently reaches the immediate effective area of the second magnetic element 302, whose field 304 will again force the magnetic actuator 102, by a rotation, to orient itself according to its magnetic field 304 oriented, with respect to the circular path of the chamber, opposite to the field 303 of the first magnetic element. Thus, the magnetic actuator 102 experiences, by the rotation of the chamber 101 and the opposite polarity of the magnetic elements 301, 302 outside the chamber, a rotation in addition to the translational movement based on a magnetic attraction to the magnetic elements 301, 302.

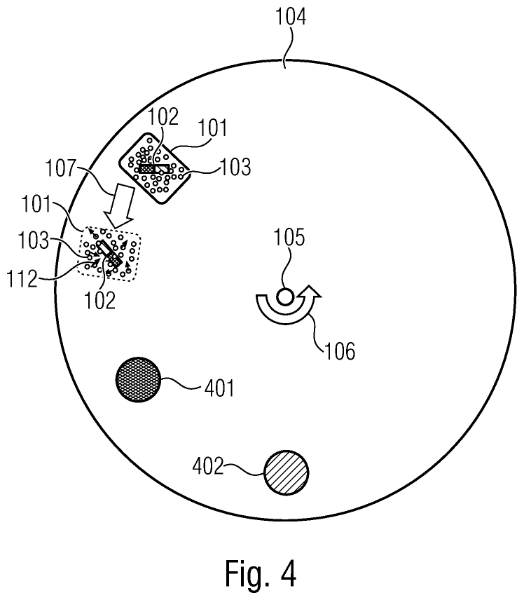

FIG. 4 shows a schematic top view of an example of an embodiment of the magnetic elements of the lysis apparatus. The illustration corresponds to FIG. 1a, with the exception of the arrangement and the type of the magnetic elements outside the chamber. Therefore, the same elements are provided with the same reference numbers. The example includes two magnetic elements 401, 402 that have a different angular arrangement compared to FIG. 1a and additionally only represent individual magnetic poles. The magnetic element 401 represents a north pole, the magnetic element 402 represents a south pole.

Thus, FIG. 4 is to illustrate a further embodiment of the oppositely polarized magnetic elements. By rotation of the carrier 104, the chamber 101 mounted on the carrier encounters the first magnetic element 401 representing a north pole. The magnetic actuator 102 experiences translation due to the magnetic attraction and a rotational movement, such that the south pole of the actuator orients itself in the direction of the magnetic element. After passing the magnetic element 401, with respect to the direction of movement of the chamber, the magnetic element 401, to which the south pole of the actuator has oriented itself, is located behind and no longer in front of the chamber. Accordingly, the magnetic actuator 102 will rotate, such that again the south pole of the magnetic actuator is oriented to the magnetic element 401. During the further rotation of the chamber, the same encounters the second magnetic element 402 representing a south pole opposite to the polarity of the first magnetic element 401. By rotating past the second magnetic element 402, in this case, the north pole of the magnetic actuator will orient itself to the second magnetic element, whereby the magnetic actuator 102 again experiences a rotational movement. By the rotation of the chamber 101, the magnetic actuator 102 experiences, apart from the translation by the magnetic attraction, a sequence of rotational movements, such that the same rotates and hence supports the lysis of the sample.

Such an arrangement of magnetic elements 401, 402 can consist, for example, of magnets whose respective second pole is so far apart from the chamber that this respective second pole can be neglected with regard to its influence on the magnetic actuator. A further option would be the usage of bent magnets, e.g., in the form of horseshoe magnets, such that the two poles of each magnet outside the chamber can be approximately approximated as magnetic single-poles in the planes of rotation with respect to their influence on the chamber. In this implementation, the rest of the bent magnets could be located, e.g., above or below the arrangement. Another option of such a structure is the configuration of the magnetic elements 401, 402 as rod magnets, wherein the connecting line between north pole and south pole is perpendicular to the plane of rotation, such that only one pole each is shown in FIG. 4.

These possible arrangements of the external magnetic elements or, e.g., magnets with different polarity relative to the chamber or lysis chamber are only to be considered as examples for illustrating some aspects of the disclosure and this is in no way a limiting list. In particular, arrangements with more than two magnetic elements as well as diverse angular arrangements of the at least two magnetic elements as well as further positional orientations of the magnetic elements with respect to the rotational circular path are also part of the present application in a sense of an obvious variation by a person skilled in the art. In particular, the illustrations should not represent any limitations regarding the shape of the magnetic elements. The magnetic elements can, for example, have round, square or rectangular cross sections. The same applies for the design of the magnetic actuator.

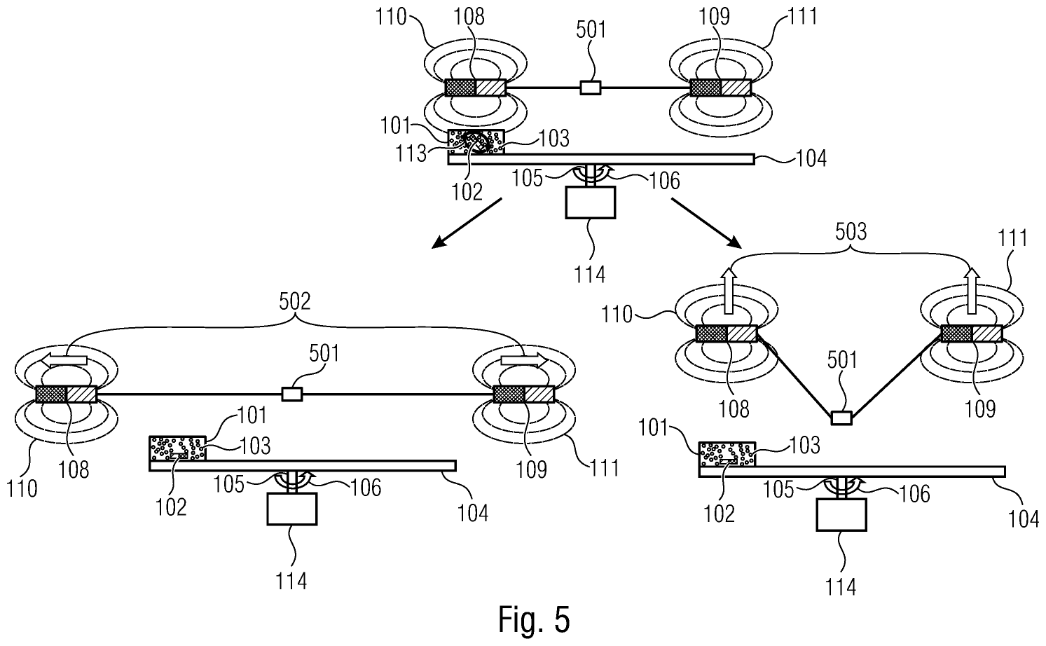

FIG. 5 shows schematic side views of two examples of the lysis apparatus with actuating means for influencing the magnetic field on the magnetic actuator independent of the rotational relative movement.

The illustrations correspond to FIG. 1b, with the exception of the actuating means and the position of the magnetic elements outside the chamber with an additional translational movement independent of the rotational relative movement between the chamber and the magnetic elements. Therefore, the same elements are provided with the same reference numbers.

FIG. 5 includes actuating means 501 connected to the magnetic elements 108, 109. The initial arrangement is shown in FIG. 5 at the top. FIG. 5 bottom left shows an option of the translational movement 502 of the magnetic elements in parallel to the plane of rotation. FIG. 5 bottom right shows an option of the translational movement 503 of the magnetic elements orthogonally to the plane of rotation.

By actuating means 501, the magnetic elements 108, 109 can be moved translationally independent of the rotational relative movement between the chamber 101 and the magnetic elements 108, 109. FIG. 5 bottom left shows a movement 502 of the magnetic elements 108, 109 in parallel to the plane of rotation. FIG. 5 bottom right shows a movement 503 of the magnetic element 108, 109 orthogonal to the plane of rotation. By both variations, the magnetic elements can be moved away so far from the rotating chamber that the magnetic interaction between the magnetic elements 108, 109 and the magnetic actuator is attenuated to such an extent that the lysis is stopped.

By the translational movement of the magnetic elements independent of the rotational movement of the chamber, the lysis can be stopped or also, e.g., started, increased or attenuated. By the actuating means 501, influencing the lysis is possible, such that with such an arrangement also, e.g., further steps of sample preparation or sample analysis can be performed, for which, e.g., rotation of the chamber is needed but no lysis is to take place.

Above that, further translational directions of movement of the magnetic elements are possible, e.g., moving the magnetic elements away in a specific angular range, e.g., at an angle that lies between the illustrated directions of movement, i.e., between orthogonal and parallel to the plane of rotation.

In other words, examples of the disclosure are based on the idea that by rotating a carrier, for example a disk, a magnetic actuator experiences an alternating magnetic field as soon as the same passes below one of the external magnetic elements, e.g. below static magnets. By the differing polarity of the external magnetic elements, the magnetic actuator experiences a translation and rotational movement. Thereby, lysis particles can collide and thereby lyse microorganisms (for example viruses, bacteria, fungi, parasites) by friction, impact and shearing forces.

In examples, after the microorganisms have been lysed, the external, e.g., static magnets can be removed by moving away a holder (e.g. according to FIG. 5) from the chamber, e.g., lysis chamber and the magnetic actuator, e.g., rod magnet. Thereby, the centrifugal and gravitational force on the magnetic actuator predominates, which stops the strong translation and rotation and hence the lysis.

Here, the present disclosure includes in particular, the spatial separation of the external, e.g., static magnetic elements from the magnetic actuator in the lysis chamber. Examples include a spatial separation by lateral translation (e.g. FIG. 5 left bottom) or spatial separation by vertical translation (e.g. FIG. 5 right bottom).

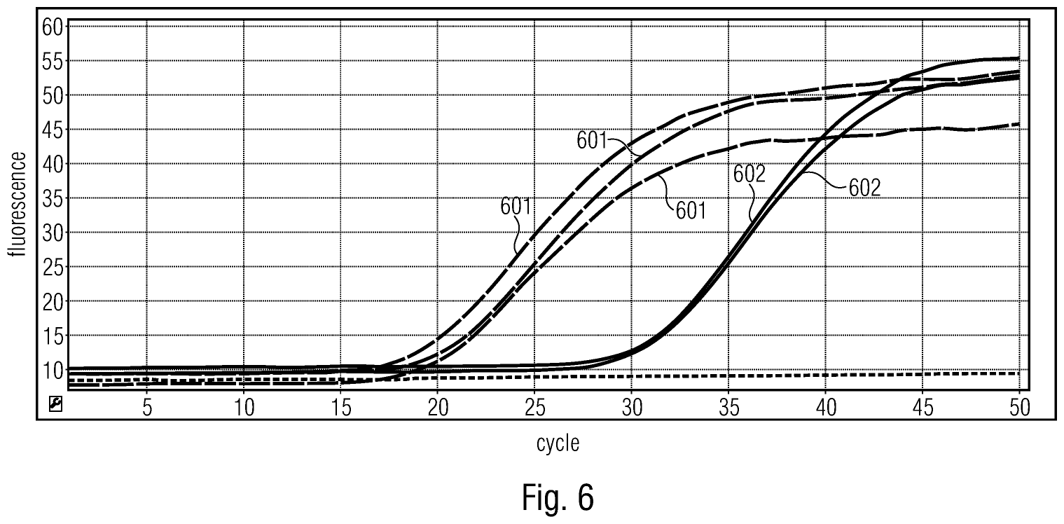

FIG. 6 shows a plot for comparing a qPCR analysis of a lysate obtained by lysis according to the present disclosure and a thermal reference lysate. Here, a measure for the result of the lysis or the fluorescence is plotted on the ordinate and a measure for the time, e.g., the cycles, is plotted on the abscissa. The plot of the qPCR analysis 601 of a lysate according to the present disclosure shows a much earlier increase of the curve with respect to the ordinate. The plot of the qPCR analysis 602 of the thermal reference lysate shows a significantly later increase of the measure for the effectivity of the lysis. The plots comprise a flattening of the curve after its respective increase. In these regions of the plots, the absolute values regarding the lysis effectivity differ only slightly for both approaches.

Accordingly, the mechanical lysis according to the present disclosure shows a much higher lysis efficiency than the thermal reference lysis. However, increasing the lysis effectivity is also possible by a suitable approach according to the present disclosure.

The results according to FIG. 6 have been obtained by using a test with a structure on a microfluidic test system comprising, as a carrier, a disk known by the name LabDisk foil disk and two permanent magnets (e.g. neodym, height 5 mm; diameter 15 mm; strength of the magnetization 45SH; type of the coating: nickel (Ni—Cu—Ni)) on a radius of 55 mm, approximately 8 mm below the carrier.

Here, glass particles (0.1 mm diameter and 0.5 mm diameter) and a magnetic actuator, e-g-, rod magnet (2 mm diameter and 3 mm height, magnetization N45, material: NdFeB, coating nickel (Ni—Cu—Ni)) were introduced into a chamber, e.g., lysis chamber.

100 μL Enterococcus faecalis in amies transport medium (by the company Copan) had been pipetted in the lysis chamber and the microfluidic carrier had been processed at a rotational speed of 20 Hz 5 min.

The lysat obtained by the lysis according to the present disclosure and a thermal reference lysat that had been processed by heating the E. faecalis in amies of the same sample to 95° C. for 5 min have subsequently been analyzed by means of qPCR. The mechanical lysis on the microfluidic carrier, e.g., disk according to the present disclosure shows a much higher lysis efficiency than the thermal reference lysis.

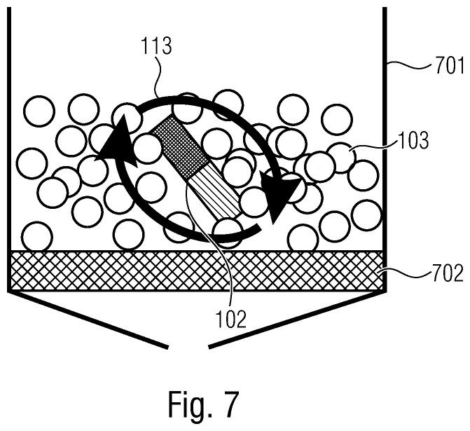

FIG. 7 shows a schematic side view of an example of a chamber 701 of a lysis apparatus including a magnetic actuator 110, lysis particles 103 as well as a diaphragm 702. The rotation of the magnetic actuator 102 during operation is indicated by arrows 113. The diaphragm 702 can be configured to enrich microorganisms of a greater volume prior to the lysis and subsequently lyse the microorganisms directly on the diaphragm 702. The diaphragm 702 can, e.g., be a filter diaphragm or, for example, a sterile filter. For example, bacteria can be enriched, e.g., on the surface of a sterile filter and thereby the same become accessible for the mechanical entry of the particles. Examples include, in particular, lysis chambers with integrated sterile filter.

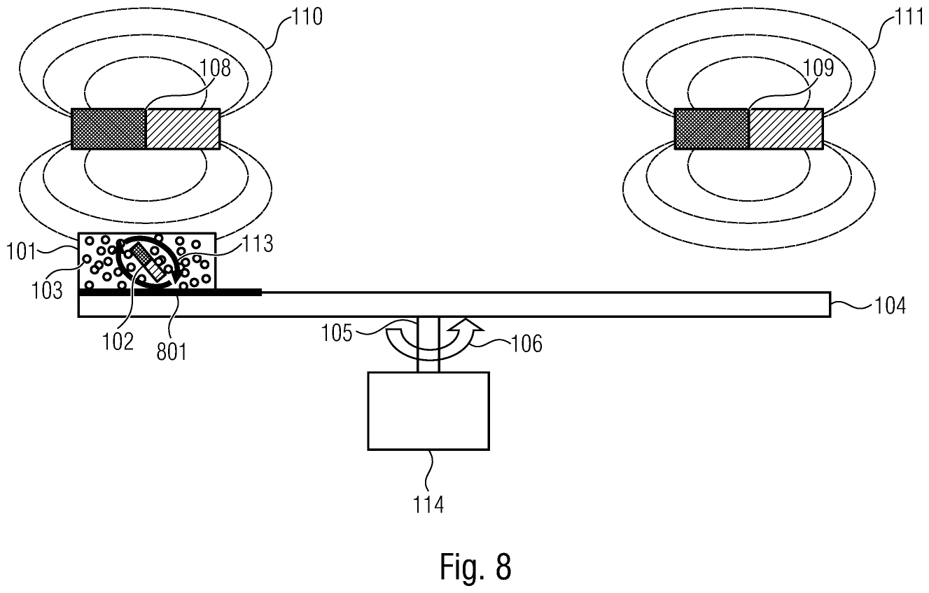

FIG. 8 shows a schematic side view of an example of the lysis apparatus with a tempering means 801. The illustration corresponds to FIG. 1b with the exception of the additional tempering means 801. The same elements are provided with the same reference numbers. The tempering means 801 is located below the chamber 102 on the carrier 104. During operation, the tempering means 801 can support the mechanical lysis by a thermal entry. The tempering means can also be used, for example, to heat the magnet within the chamber above its Curie temperature, such that the lysis can be stopped. Such switching-off of the lysis is, e.g., advantageous to provide an option to stop the lysis without additional actuating means, independent of the rotational relative movement between the chamber 101 and the magnetic elements 108, 109.

One implementation option for a tempering means is a contact heating. In examples according to the present disclosure, there is the option of positioning the lysis chamber such that a temperature entry can be realized by a contact heating. The temperature entry could be set, e.g., between the environmental temperature and up to 120° C. Thereby, the mechanical lysis can be additionally supported by a thermal entry.

In examples, the tempering means can also be a contact heating below the lysis chamber by a heating zone.

Examples of the present disclosure include two external static magnets (e.g., neodym N45) which can be positioned via a holder above or below a disk (e.g. according to FIG. 1). The polarities of the external magnets are, e.g., opposite to one another with respect to a rotating lysis chamber. This specific polarity can be realized in different ways (e.g., according to FIG. 2) and is, in examples, decisive for the rotational movement of the magnetic actuator around its own axis. During a lysis phase, the distance between the magnet and lysis chamber in z direction, e.g., vertically to the plane of rotation, can be, e.g., between 0.1 mm to 50 mm. Further, the external magnets can be positioned on radii having a maximum distance of 30 mm to the radius of the lysis chamber. Within the lysis phase, the disk can rotate, e.g., at 0.5 Hz-40 Hz. The magnetic actuator can be realized, e.g., as neodym rod magnet, for example with a magnetization N48, a length of 2 mm to 4 mm and a diameter of 2 mm to 4 mm. Further, lysis particles can be formed by glass particles having a diameter of, e.g., 0.15 mm to 0.5 mm or 0.15 mm to 0.2 mm.

Further examples according to the present disclosure include lysis apparatuses for mechanical lysis in a centrifugal microfluidic cartridge with a lysis chamber including lysis particles and a permanent magnet, at least two external magnets, wherein the external magnets are oppositely polarized relative to the magnet in the chamber, e.g., the magnet in the disk, or the lysis chamber, to effect a rotation of the magnet within the lysis chamber, wherein the size of the lysis chamber has at least the size of the length of the magnet in two spatial directions (Δr, Δφ), e.g., radial and azimuthal with respect to the plane of rotation or (Δz, Δφ), e.g., vertical and azimuthal with respect to the plane of rotation or (Δz, Δr), e.g., vertical and radial with respect to the plane of rotation (rotation in the plane around z axis or rotation around radius vector of the rotating system r).

Further examples include lysis apparatuses, wherein the external magnetic elements are located above the chamber or the disk at a distance in z direction, e.g., vertically to the plane of a rotation at a maximum of 5 cm and deviate not more than 5 cm from the radius of the internal magnetic actuator.

Further examples include lysis apparatuses, wherein the size of the lysis chamber in three orthogonal spatial directions (x,y,z) has at least approximately the length of the longest diagonal of the magnetic actuator (free rotation; slightly smaller than the magnet in z direction, e.g. vertical to the planes of rotation up to, e.g., ˜20% is also possible).

Further examples include lysis apparatuses, wherein the lysis takes place at a (continuous) rotational frequency of at least 2 Hz. Further examples include lysis apparatuses wherein the lysis takes place at a (continuous) rotating frequency of a maximum of 30 Hz. Further examples include lysis apparatuses, wherein the chamber includes lysis particles of a size of <0.5 mm. Further examples include lysis apparatuses, wherein switching off the lysis by moving away the external magnetic elements is possible. Further examples include lysis apparatuses, wherein the external magnetic elements are arranged at an angle of 20-180°. Further examples include lysis apparatuses, wherein temperature entry into the lysis chamber can be implemented to thermally support the lysis function.

### Advantages of the Present Disclosure

The induced translational and rotational movement of the magnets within the chamber, e.g. a magnetic actuator by different polarity of the external, e.g. static magnetic elements with respect to the chamber, e.g. lysis chamber results in a much better mixture of the sample in the chamber in particular with particles in the chamber. Thus, in the statistical mean, a larger part of the microorganisms can be subject to friction, impact and shear movements. Thereby, very efficient lysis can be realized. In molecular diagnostic (quick) tests, the factor “time to result”, which includes the time needed for sample preparation and analysis is a decisive factor. By efficient and fast lysis, a lot of time can be saved in this essential step.

By the described arrangement of external magnetic elements and the chamber, e.g., the lysis chamber, a lysis apparatus according to the present disclosure with respective configuration needs only very little space in radial and azimuthal direction. As the space and radial azimuthal direction on a microfluidic test carrier is very valuable to achieve an overall integration of sample preparation and analysis, this represents a further advantage compared to conventional technology.

Although some aspects of the present disclosure have been described as features in the context of an apparatus, it is obvious that such a description could also be considered as a description of the corresponding method features. Although some aspects have been described as features in the context of a method, it is obvious that such a description could also be considered as a description of the corresponding features of an apparatus or the functionality of an apparatus.

In the previous detailed description, partly, different features have been grouped together in examples to rationalize the disclosure. This type of disclosure is not to be interpreted as the intention that the claimed examples comprise more features than explicitly stated in each claim. Rather, as shown by the following claims, the subject matter consist of less than all features of an individual disclosed example. Consequently, the following claims are incorporated in the detailed description, wherein each claim can be its own separate example. While each claim can be its own individual separate example, it should be noted that although dependent claims in the claims relate to a specific combination with one or several other claims, other examples also include a combination of dependent claims with the subject matter of each other dependent claim or a combination of each feature with other dependent or independent claims. Such combinations are included, except where it is stated that a specific combination is not intended. Further, it is intended that also a combination of features of a claim is included in each other independent claim, even when this claim is not directly dependent on the independent claim.

While this invention has been described in terms of several advantageous embodiments, there are alterations, permutations, and equivalents, which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

### REFERENCES

[1] Kido, Horacio, et al. “A novel, compact disk-like centrifugal microfluidics system for cell lysis and sample homogenization.” Colloids and Surfaces B: Biointerfaces 58.1 (2007): 44-51.
[2] Siegrist, Jonathan, et al. “Validation of a centrifugal microfluidic sample lysis and homogenization platform for nucleic acid extraction with clinical samples.” Lab on a Chip 10.3 (2010): 363-371.

1. Lysis apparatus, comprising:
a chamber for receiving a sample;
at least one magnetic actuator located within the chamber;
at least two magnetic elements arranged outside the chamber and
a driving unit for effecting a rotational relative movement between the chamber and the magnetic elements arranged outside the chamber, by which the chamber successively passes the magnetic elements located outside the chamber, wherein the polarity of the magnetic elements is opposite with respect to the circular path of the rotational relative movement, such that the magnetic actuator arranged within the chamber is moved both translationally and rotationally around an own axis of the magnetic actuator to effect lysis of the sample,
wherein the chamber is configured to enable the at least one magnetic actuator located within the chamber to move both translationally and rotationally around an own axis of the magnetic actuator.

2. Lysis apparatus according to claim 1, wherein the magnetic elements are magnetic poles or wherein each magnetic element is a magnet.

3. Lysis apparatus according to claim 1, which is configured to at least reduce the magnetic field acting on the magnetic actuator arranged within the chamber from the at least two magnetic elements arranged outside the chamber, independent of the rotational relative movement, and
further comprising an actuator to change the distance between the chamber and the at least two magnetic elements arranged outside the chamber, independent of the rotational relative movement.

4. Lysis apparatus according to claim 3, wherein the actuator is configured to move the at least two magnetic elements arranged outside the chamber perpendicular to the plane of rotation.

5. Lysis apparatus according to claim 3, wherein the actuator is configured to move the at least two magnetic elements arranged outside the chamber parallel to the plane of rotation, independent of the rotational relative movement

6. Lysis apparatus according to claim 1, wherein the at least two magnetic elements arranged outside the chamber are controllable and/or variable electromagnets.

7. Lysis apparatus according to claim 6, which is configured to at least reduce the magnetic field acting on the magnetic actuator arranged within the chamber from the at least two magnetic elements arranged outside the chamber, independent of the relative rotational motion.

8. Lysis apparatus according to claim 1, wherein the lysis apparatus comprises at least one lysis particle located within the chamber.

9. Lysis apparatus according to claim 8, wherein the at least one lysis particle comprises maximum dimensions of less than 0.5 mm.

10. Lysis apparatus according to claim 1, wherein the at least two magnetic elements arranged outside the chamber are configured to be stationary at the time of the lysis and the driving unit is configured to rotate the chamber with respect to an axis of rotation relative to the magnetic elements located outside the chamber.

11. Lysis apparatus according to claim 1, wherein the chamber comprises a diaphragm.

12. Lysis apparatus according to claim 1, wherein the lysis apparatus comprises tempering unit configured to change the temperature of the chamber.

13. Lysis apparatus according to claim 1, wherein the at least two magnetic elements outside the chamber are arranged at an angle in the plane of rotation of 20° to 180° to each other.

14. Lysis apparatus according to claim 1, wherein the driving unit is configured to provide the rotational relative movement with a rotational frequency of 0.5 Hz to 40 Hz, advantageously of 2 Hz to 30 Hz.

15. Lysis apparatus according to claim 1, wherein the chamber comprises at least the length of the longest diagonal of the magnetic actuator located within the chamber, within two of three spatial directions that are perpendicular to each other, and comprises at least the length of the longest diagonal of the magnetic actuator located within the chamber minus 20% in a third of the three spatial directions perpendicular to each other.

16. Lysis apparatus according to claim 1, wherein the chamber comprises at least the size of the length of the magnetic actuator located within the chamber in at least two directions of three directions that are formed by the direction of the axis of rotation, the radial direction with respect to the rotation and the azimuthal direction with respect to the rotation.

17. Lysis apparatus according to claim 1, wherein the at least two magnetic elements outside the chamber are configured to comprise, at the time of the lysis, a maximum perpendicular distance with respect to the plane of the rotational relative movement to the chamber that is 5 cm maximum and/or
wherein the at least two magnetic elements outside the chamber are configured to comprise, at the time of the lysis, a maximum radial distance from the magnetic actuator located within the chamber of 5 cm.

18. Lysis apparatus according to claim 1, wherein the lysis apparatus comprises tempering unit configured to heat the at least one magnetic actuator located within the chamber above the Curie temperature to deactivate the at least one magnetic actuator.

19. Lysis method, comprising:
introducing a sample into a chamber, wherein at least one magnetic actuator is located in the chamber and wherein the chamber is configured to enable the magnetic actuator located within the chamber to move both translationally and rotationally around an own axis of the magnetic actuator;
effecting a rotational relative movement between the chamber wherein the sample and at least one magnetic actuator are located, and between at least two magnetic elements located outside the chamber, wherein the polarity of the at least two magnetic elements outside the chamber is opposite with respect to the circular path of the rotational relative movement, such that the magnetic actuator located within the chamber is moved both translationally and rotationally around an own axis of the magnetic actuator to effect lysis of the sample.

20. Lysis method according to claim 19, wherein at least one lysis particle is introduced into the chamber.

21. Lysis method according to claim 19, wherein the temperature of the sample is changed.

22. Lysis method according to claim 19, wherein the at least two magnetic elements arranged outside the chamber are moved independent of the rotational relative movement to at least reduce the magnetic field acting on the magnetic actuator located in the chamber.

23. Lysis method according to claim 19, wherein the at least two magnetic elements arranged outside the chamber are controllable and/or variable electromagnets and wherein the at least two electromagnets arranged outside the chamber are controlled or regulated such that the magnetic field acting on the magnetic actuator arranged within the chamber is at least reduced, independent of the rotational relative movement.

24. Lysis method according to claim 19, wherein the rotational frequency of the rotational relative movement is adjusted between 0.5 Hz and 40 Hz, advantageously between 2 Hz and 30 Hz.
